(19) United States
(12) Patent Application Publication
(10) Pub. No.: US 2020/0323657 A1
(43) Pub. Date: Oct. 15, 2020

(54) Systems And Methods For Robotic Mirror Therapy Of The Hand

(71) Applicant: Board of Regents, The University of Texas System, Austin, TX (US)

(72) Inventors: Charu Pande, Bhiwadi (IN); Mahdi Haghshenas-Jaryani, Las Cruces, NM (US); Muthu Wijesundara, Fort Worth, TX (US)

(21) Appl. No.: 16/849,914

(22) Filed: Apr. 15, 2020

Related U.S. Application Data
(60) Provisional application No. 62/833,892, filed on Apr. 15, 2019.

(51) Int. Cl. A61F 2/70; A61F 2/58

(52) U.S. Cl. CPC A61F 2/70; A61F 2002/6827; A61F 2002/704; A61F 2/583

(57) ABSTRACT

In one embodiment, a robotic mirror therapy system for providing therapy to a user having a impaired hand, the system including a motion command glove configured to be worn on a healthy hand of the user or another person, the motion command glove comprising position sensors configured to determine the position and orientation of the healthy hand and its fingers, a motion actuator glove configured to be worn on the user's impaired hand, the motion actuator glove comprising actuators configured to actuate the impaired hand and its fingers, and a control unit configured to receive position data from the motion command glove and to control actuation of the actuators of the motion actuator glove such that the impaired hand mirrors motion of the healthy hand.

### CROSS-REFERENCE TO RELATED APPLICATION

This application claims priority to co-pending U.S. Provisional Application Ser. No. 62/833,892, filed Apr. 15, 2019, which is hereby incorporated by reference herein in its entirety.

### BACKGROUND

About two thirds of stroke survivors live with impaired arm and hand functions. Therapeutic interventions leverage the brain's inherent neuroplasticity to increase adaptation to stroke by using both sensory and motor stimuli through repetitive motions.

Mirror therapy is one of the crucial therapy modalities used in stroke rehabilitation. In mirror therapy, a mirror is placed in the patient's sagittal plane so that the patient cannot see the paretic limb. The patient is then asked to move the healthy limb while observing it in the mirror. The imagery and visual feedback from the mirror create the impression in the patient's mind that their paretic limb is moving. This kind of therapy is known to activate the sensorimotor cortex and facilitate the brain neuroplasticity that aid in adaptation to stroke.

Inspired by the success of mirror therapy, a new form of therapy using assistive robotic systems has been developed to further facilitate motor recovery. This therapy is typically referred to as robotic bilateral therapy, robotic bimanual therapy, or robotic mirror therapy. With this therapy, the motion of a healthy limb is used to create the same motion of the paretic limb. Robotic mirror therapy applies mirrored motion of the healthy limb to the paretic limb, in contrast to the conventional mirror therapy where the paretic limb remains inactive during treatment. The main objective of the robotic mirror therapy is to provide proprioceptive stimulus to the sensory cortex to facilitate neuroplasticity and functional recovery. This therapy modality also addresses one of the deterministic factors, i.e., nonspontaneous or limited spontaneous use of the paretic limb that affects full recovery after stroke. Applying motion to the paretic limb also helps reduce contracture, maintain muscle tone, and lower spasticity. Furthermore, as the patient can direct the therapy by himself or herself, robotic mirror therapy addresses major limiting factors of the conventional therapist-centered rehabilitation, including rising costs and shortages of available resources, such as facilities, therapists, and funding.

While robotic mirror therapy has great potential in treating stroke victims, few have attempted to assist finger motion in hand rehabilitation. The systems that have been developed have various shortcomings, such as an inability to operate in three-dimensional space, limitations in grasping and manipulating objects in real-world scenarios, and lack of fine control of the fingers. It, therefore, can be appreciated that it would be desirable to have an effective system and method for robotic mirror therapy of the hands.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a schematic diagram of an embodiment of a robotic mirror therapy system.

FIG. 2A is a plan view of a dorsal side of an embodiment of a motion command glove shown in FIG. 1.

FIG. 2B is a plan view of a palmar side of the motion command glove of FIG. 2A.

FIG. 3A is a plan view of a dorsal side of an embodiment of a motion actuator glove shown in FIG. 1.

FIG. 3B is a plan view of a palmar side of the motion actuator glove of FIG. 3A.

FIG. 4 is a block diagram illustrating an embodiment of a control process that can be performed by the robotic mirror therapy system of FIG. 1.

FIG. 5 is a block diagram that illustrates an example implementation of a non-adaptive hybrid force-position control algorithm that can be executed by the robotic mirror therapy system of FIG. 1.

FIG. 6 is a block diagram that illustrates an example implementation of an adaptive hybrid force-position control algorithm that can be executed by the robotic mirror therapy system of FIG. 1.

FIG. 7 is a screen shot of an embodiment of a graphical user interface that can be displayed by the robotic mirror therapy system of FIG. 1.

FIG. 8 is a graph that plots data collected from both a motion command glove and a motion actuator glove of an experimental system having a configuration similar to that of the robotic mirror therapy system of FIG. 1.

### DETAILED DESCRIPTION

As described above, it would be desirable to have an effective system and method for providing robotic mirror therapy of the hands. Disclosed herein are examples of such systems and methods. In one embodiment, a robotic mirror therapy system includes a motion command glove that is worn on the healthy hand and a motion actuator glove that is worn on the impaired (e.g., paretic or injured) hand. The motion command glove is equipped with position sensors so that the motions of the hand and fingers can be tracked. In addition, the motion command glove can be equipped with force sensors so that the forces imposed upon the hand can be measured. The motion actuator glove is also equipped with position sensors and can also comprise force sensors. In addition, the motion actuator glove includes actuators so that, when the healthy hand is moved, apply the same movement to the impaired hand. Furthermore, if the healthy hand grasps or otherwise interacts with an object, the impaired hand can be made to likewise grasp or otherwise interact with a similar object with the same amount of force. With this master-slave arrangement, therapy is provided to the impaired hand by performing actions along with the healthy hand.

In the following disclosure, various specific embodiments are described. It is to be understood that those embodiments are example implementations of the disclosed inventions and that alternative embodiments are possible. Such alternative embodiments include hybrid embodiments that include features from different disclosed embodiments. All such embodiments are intended to fall within the scope of this disclosure.

Robotic mirror therapy uses a master-slave scheme in which a robotic system senses motion of a healthy limb and controls an impaired limb in a manner that “mirrors” the motion of the healthy limb. As noted above, various attempts have been made at developing such a system for the hands, but those systems have various shortcomings that limit their usefulness. By considering technology gaps and the high incidence of hand disability and injuries, a novel hand therapy robotic mirror therapy system has been developed. In one embodiment, the system comprises a motion command glove, a motion actuator glove, a control unit, and a graphical user interface. The motion command glove, which is worn on the healthy hand, can comprise embedded position and force sensors configured to acquire joint trajectories and contact forces associated for particular movement, grasping, and manipulation actions. The motion actuator glove, acting under the control of the control unit, provides fine motor control over the impaired hand based on the inputs from the motion command glove. The graphical user interface facilitates the entry of therapy inputs, therapy data visualization, and outcome data logging for therapy evaluation.

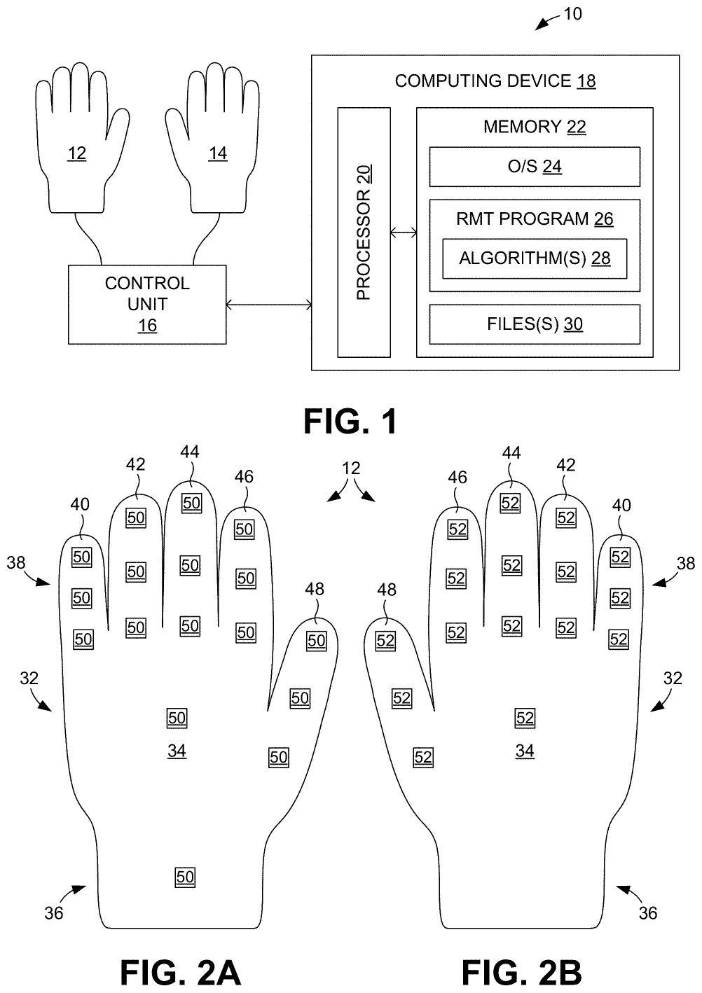

FIG. 1 illustrates an example of a robotic mirror therapy system 10 that can be used to facilitate robotic mirror therapy. As shown in this figure, the system 10 generally comprises a motion command glove (MCG) 12 and a motion actuator glove (MAG) 14 that are configured to be worn by a user (e.g., patient), a control unit 16, and a computing device 18. The MCG 12 (whether it be a left-hand glove or a right-hand glove) is configured to be worn on the user's healthy hand and the MAG 14 (whether it be a left-hand glove or a right-hand glove) is configured to be worn on the user's impaired hand. While the MCG 12 has been described as being worn on the user's healthy hand, it is noted that the MCG could instead be worn on a healthy hand of another person providing guided therapy to the user, such as a physical therapist. Each of the gloves 12, 14 is in electrical communication with the control unit 16, which is in turn in electrical communication with the computing device 18. Although the gloves 12, 14 and computing device 18 are shown connected to the control unit 16 with wires, it is noted that a wireless communication scheme could be used between the gloves and the control unit, the control unit and the computing device, or both.

As described in greater detail below, the control unit 16 is configured to receive and execute commands from the computing device 18 that control the manner in which the system 10 is operated. More particularly, the control unit 16 receives sensed data regarding movement of a healthy hand and, responsive to commands from software that are used to control manipulation of the user's impaired hand.

With further reference to FIG. 1, the computing device 18 generally comprises a processor 20 and memory 22. The processor 20 is configured to execute programs stored within the memory 22, which is a non-transitory computer-readable medium can comprise a volatile storage medium, non-volatile storage medium, or both. Stored within memory 22 is an operating system 24 that is configured to control overall operation of the computing device 18, and a robotic mirror therapy (RMT) program 26 that is configured to control the operation of the robotic mirror therapy system 10, collect and store data sensed by the system, analyze the collected data, issue control commands for the control unit 16 for the purpose of controlling manipulation of the MAG 14, and present collected data and results of the analysis. As discussed below, the program 26 can comprise one or more algorithms (comprising computer-executable instructions) that are configured to analyze the data. Also stored within memory 22 are one or more files 30 in which the collected data and the results of the analysis can be stored in relation to the user. Although the control unit 16 and the computing device 18 have been illustrated as being separate components, it is noted that, in other embodiments, the two components can be integrated into a single control component. Alternatively, one or more of the functionalities of the computing device 18 can be performed by the control unit 16. For example, if the control unit 16 has sufficient computing power, it can execute the robotic mirror therapy (RMT) program 26.

Turning to FIGS. 2A and 2B, illustrated is an example embodiment for the MCG 12. Beginning with FIG. 2A, which shows a dorsal (outer) side of the MCG 12, the MCG comprises a glove 32 that includes a body 34 configured to surround the body of the hand, including the palm and the top of the hand. Extending from a bottom edge of the body 34 is a wrist cuff 36 and extending from top and side edges are finger sleeves 38. The finger sleeves 38 include a pinky finger sleeve 40, a ring finger sleeve 42, a middle finger sleeve 44, an index finger sleeve 46, and a thumb sleeve 48 (the thumb being a “finger”). The glove 32 is made of a thin, flexible material, such as a natural or synthetic fabric that can, in some embodiments, be an elastic fabric that fits the contours of the user's hand.

With particular reference to FIG. 2A, integrated into the dorsal side of the MCG 12 are multiple position sensors 50 that are configured to detect and track motion of the user's wrist, hand, and fingers while wearing the MCG. The position sensors 50 can comprise any sensors that can be used to sense/track motion of the healthy hand. In some embodiments, the position sensors 50 comprise inertial measurement units (IMUs) that are configured to measure each finger segment's, the hand's, and the wrist's motion and orientation. Such IMUs can comprise one or more of an accelerometer, a gyroscope, and a magnetometer. In some embodiments, each IMU can be associated with a different segment of each finger so as to capture the angular motions of the finger segments and, consequently, the angular motion (rotation) of the joints. In other embodiments, the position sensors 50 can comprise flex sensors that are configured to measure the amount of deflection or bending of the fingers.

Regardless of the specific nature of the position sensors 50, the sensors can be used to track manipulation of the healthy hand for the purpose of manipulating the impaired hand in the same manner. As noted above, the position sensors 50 are integrated into the MCG 12. In some embodiments, the position sensors 50 can be attached to the inner or outer surface of the glove material, or can be located between opposed layers of the glove material. Although not illustrated in FIG. 2A, one or more wires extend from each of the position sensors 50 to the control unit 16 (FIG. 1). Furthermore, while the position sensors 50 have been described as being integrated into the dorsal side of the glove 32, it is noted that, in other embodiments, one or more of the position sensors can be integrated into the palmar side of the glove.

Referring next to FIG. 2B, illustrated is a palmar (inner) side of the MCG 12. Integrated into the palmar side of the glove 32 are multiple force sensors 52 that are configured to measure forces applied to discrete parts of the healthy hand. Such forces can result from some form of interaction of the hand with one or more objects. For example, the forces can result from the healthy hand grasping an object, such as a cup.

The force sensors 52 can comprise any sensors with which the applied forces can be determined. In some embodiments, the force sensors 22 each comprise a capacitive, piezoelectric, piezoresistive, or resonant-based sensor. As with the position sensors 50, the force sensors 52 can be attached to the inner or outer surface of the glove material, or can be located between opposed layers of the glove material. Although not illustrated in FIG. 2B, one or more wires extend from each of the force sensors 52 to the control unit 16 (FIG. 1).

Although the MCG 12 has been described as comprising a conventional glove, it is noted that, in some embodiments, the glove 32 can instead be a partial glove that does not cover all of the surfaces of the hand and wrist. In fact, the glove may not actually be a “glove” in the strictest sense of the term. For example, the “glove” can comprise multiple independent finger sleeves or finger loops that are tethered to a wrist cuff or other component of the term “glove.” Regardless of the particular configuration, Applicant notes that the “glove” is used broadly to describe substantially any article that can be worn on the hand in a manner in which the various sensors described above are placed in positions that enable their above-described functionalities.

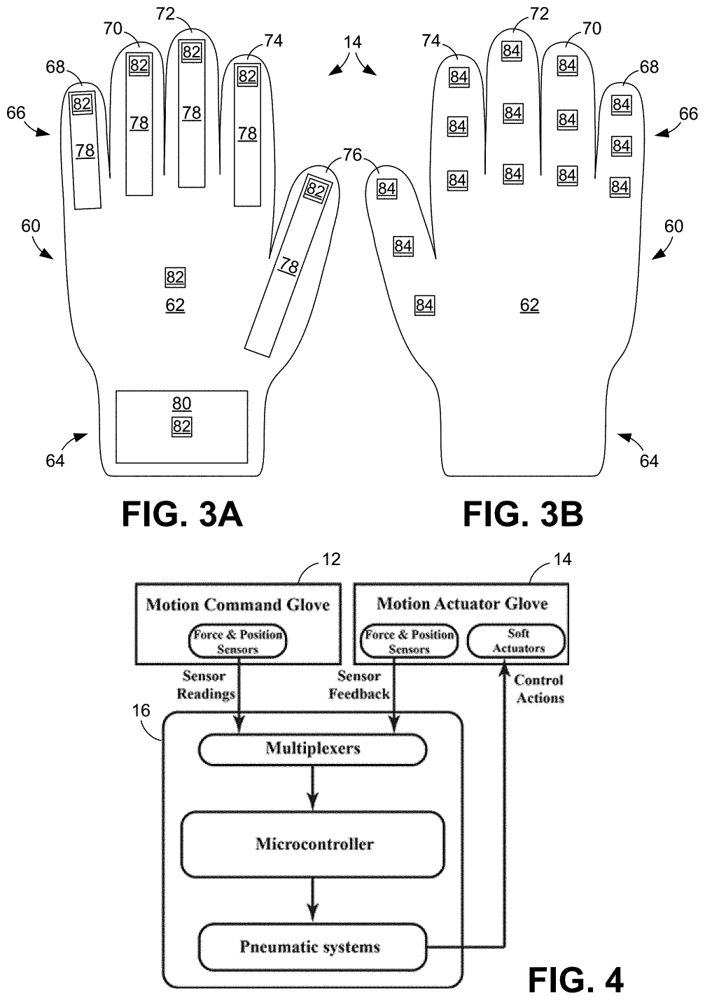

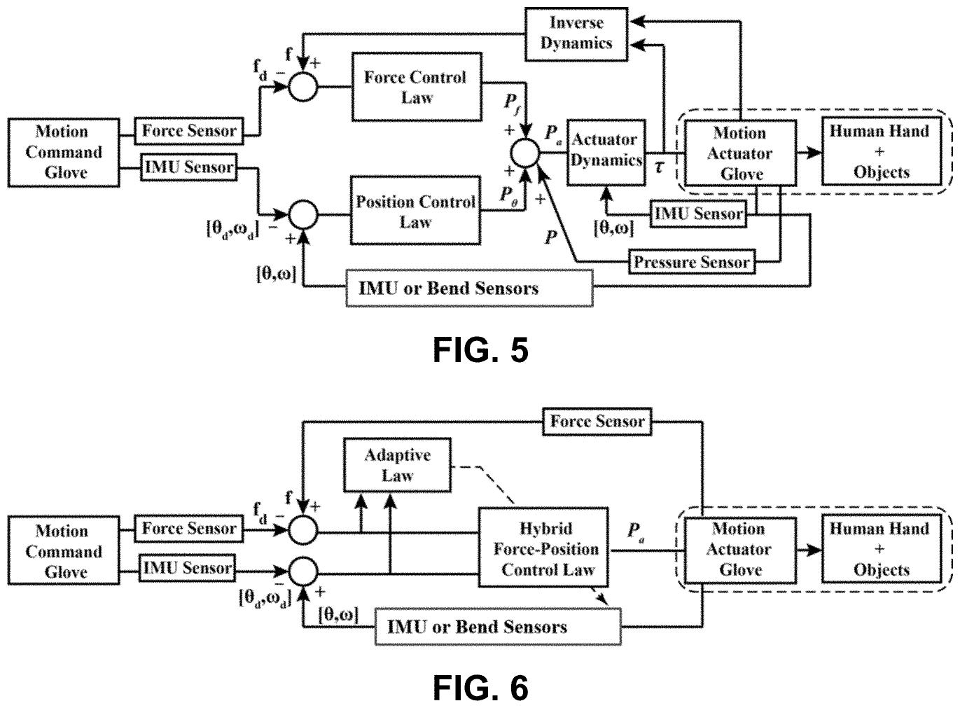

Turning next to FIGS. 3A and 3B, illustrated is an example embodiment for the MAG 14. Beginning with FIG. 3A, which shows a dorsal (outer) side of the MAG 14, the MAG also comprises a glove 60 that includes a body 62, a wrist cuff 64, and finger sleeves 66. The finger sleeves 66 include a pinky finger sleeve 68, a ring finger sleeve 70, a middle finger sleeve 72, an index finger sleeve 74, and a thumb sleeve 76.

Integrated with the dorsal side of the MAG 14 are multiple finger actuators 78 that are configured to actuate an associated finger of the user's impaired hand. More specifically, the finger actuators 78 can bend the fingers inward toward the palm, as when the hand is used to grasp an object, as well as extend the fingers outward away from the palm, as when the hand releases an object. In addition to finger actuators 78, the MAG 14 can include a wrist actuator 80, which is configured to flex and extend the hand at the wrist.

In some embodiments, the various actuators 78, 80 are “soft” fluidic actuators that extend and retract responsive to the pressure of a fluid present within the actuators. Such actuators may be preferred over other types of actuators as they enable soft robotic manipulation of the impaired hand as opposed to rigid control, as would be provided by an exoskeleton or other rigid mechanism. Examples of fluidic actuators include pneumatic actuators that use air as a driving fluid and hydraulic actuators that use oil as a driving fluid. In some embodiments, the actuators 78, 80 can be configured in similar manner to the actuators disclosed in commonly-assigned U.S. Patent Application No. US 2018/0303698, which is hereby incorporated by reference into the present disclosure.

The actuators 78, 80 can be attached to the inner or outer surface of the glove material, or can be located between opposed layers of the glove material, depending upon the configuration and nature of the actuators. Although not illustrated in FIG. 3A, one or more wires extend from each of the of the actuators 78, 80 to the control unit 16 (FIG. 1). Furthermore, while the actuators 78, 80 have been described as being integrated with the dorsal side of the glove 60, it is noted that, in other embodiments, one or more of the actuators can be integrated with the palmar side of the glove or on the sides of the fingers/wrist.

With further reference to FIG. 3A, the dorsal side of the MAG 14 can further comprise multiple position sensors 82 that are configured to detect and track motion of the user's wrist, hand, and fingers while wearing the MAG. The position sensors 82 can have configurations similar to the position sensors 50 described above in relation to MCG 12. While the position sensors 82 have been described as being integrated into the dorsal side of the glove 60, it is noted that, in other embodiments, one or more of the position sensors can be integrated with a palmar side of the glove.

Referring next to FIG. 3B, illustrated is the palmar (inner) side of the MAG 14. Integrated into the palmar side of the glove 60 are multiple force sensors 84 that can also have configurations similar to the position sensors 82 described above. Although not illustrated in FIG. 3B, one or more wires extend from each of the force sensors 84 to the control unit 16 (FIG. 1).

While the MAG 14 has also been described as comprising a conventional glove, it is noted that, in some embodiments, the glove 60 can also be a partial glove that does not cover all of the surfaces of the hand and wrist. Regardless of the particular configuration, Applicant reiterates that the term “glove” is used broadly to describe substantially any article that can be worn on the hand in a manner in which the various actuators and sensors described above are placed in positions that enable their above-described functionalities.

When the system 10 is used to provide robotic mirror therapy, the healthy hand's movements are used as a reference for manipulation of the MAG 14. This movement is tracked by the position sensors 50 of the MCG 12. In addition, the forces encountered by the healthy hand are tracked by the force sensors 52 of the MCG 12. The data collected by the sensors 50, 52 are transmitted to the control unit 16. This data can then be analyzed to determine how to actuate the MAG 14 so as move the impaired hand in a manner in which it mirrors the movement of the healthy hand. This analysis can be performed by the robotic mirror therapy program 26 (FIG. 1), whether it is executed by the control unit 16 or the computing device 18. In the latter case, the data is first transmitted from the control unit 16 to the computing device 18.

As discussed above, the robotic mirror therapy program 26 comprises one or more algorithms that can be used to analyze the collected data by the MCG 12 and determine how to manipulate the MAG 14. In some embodiments, a non-adaptive or an adaptive hybrid force-position control algorithm is used to determine the control actions that are needed to manipulate the MAG 14 in a manner in which mirrored movement of the impaired hand is achieved. In cases in which the MAG 14 is fluid-based, this can entail determining actuation pressures and/or fluid flows necessary for correct actuation of the finger and/or wrist actuators 78, 80. The control actions determined by the control algorithm can be provided to the control unit 16, which can then control the actuators 78, 80 as commanded.

In some embodiments, the data collected from both the MCG 12 and the MAG 14, the data generated by the algorithm (e.g., control data), as well as the results of data analysis can be presented to the user and/or a medical professional (e.g., physical therapist) in a graphical user interface shown in a display device of the control unit 16 or the computing device 18. In addition, the data/results can be stored within one or more of the files 30 in the memory 22. The collected and generated data can include finger trajectories, the degree and manner of assistance provided by the MAG 14, as well as force and/or pressure data. The data that is collected/generated during each rehabilitation session can be provided as feedback to the user and/or medical professional and the user's progress can be tracked over time. In addition, retrospective analyses of the therapy's effectiveness can be performed using the stored data.

FIG. 4 illustrates an example control process that can be used by the robotic mirror therapy system 10. In this example, it is assumed that the MAG 14 uses pneumatic actuators to manipulate the impaired hand. As shown in the figure, the control unit 16 can comprise one or more multiplexers, a microcontroller, and one or more pneumatic systems that include various pneumatic components, which can include one or more pumps, valves (e.g., solenoid valves and/or proportional valves), pressure sensors, and vacuum sensors. The motion and force sensors 50, 52 of the MCG 12 can be interfaced with the microcontroller 34 via the multiplexer 36. These sensors 50, 52 can communicate with the microcontroller 34 either directly or over a serial bus, such as a I2C bus. The operation of the pneumatic system is controlled by the microcontroller 34 and supporting circuitry.

The pneumatic components drive pressurized air into and out of the actuators 78, 80 to provide the necessary hand movements, such as finger flexion and/or extension and wrist flexion and/or extension. The capability of applying both pressure and vacuum enables bidirectional motion regardless of joint stiffness of the hand. In some embodiments, the air pressure is controlled using proportional valves by varying their orifice size using pulse width modulation (PWM). The pressure and vacuum in the actuators 78, 80 can be monitored using pressure and vacuum sensors, respectively. Data from these sensors provides feedback to the control unit 16, which executes controlled actions by controlling airflow using the proportional and solenoid valves.

FIG. 6 illustrates a non-adaptive hybrid force-position control scheme. In this control scheme, proportional-derivative (PD) position and force control algorithms are used to control joint position based upon changing the fluid or airflow from the pneumatic system. The joint space position control enables tracking of the joint trajectories, angular velocities, and interaction forces of the MCG 12 and, therefore, the healthy hand. These trajectories, angular velocities, and forces are compared against the sensor feedback from the MAG 14 and then the error is used in the closed-loop PD position and force control algorithms to determine a control action. In some embodiments, proportional-integral (PI) position and force control algorithms can be used in lieu of the PD position and force control algorithms.

The adaptive force-position control scheme is based on hybrid force-position techniques with an integrated adaptive controller to generate the required motion and force for the user's impaired hand by following desired trajectories and contact forces received from the healthy hand for rehabilitation-based grasping tasks and hand gestures. FIG. 6 illustrates an example of operation of the algorithm. The adaptive force-position control algorithm controls both position and contact forces. As is indicated in that figure, the algorithm comprises two control loops: (1) a position control loop at the joint space and (2) a force control loop in the operation space with desired angular displacement, angular velocity, and contact force as the control parameters.

The joint space position control enables the impaired digits to follow desired (healthy hand) joint trajectories, including angular position (θ_{d}) and angular velocity (ω_{d}). These desired joint trajectory inputs are compared against the MCG 12 feedback and then the error is used in a closed-loop proportional-integral-derivative (PID) position control algorithm to determine a control action. Concurrently, the force control loop ensures that a desired contact force measured between the fingers or the fingers and another object is met by considering motion and dynamic coupling between the MAG 14 and the user's hand along with interaction with objects. The contact forces between the robotic digit and the finger (f) is compared against the desired input force (f_{d}), and then the error is input into a proportional force control law in order to determine another control action. The contact force can be calculated in a feedback loop using an inverse dynamic model that receives the angular position feedback from the IMU sensors and the soft actuation torque (τ) from an actuator dynamic model.

In order to calculate the applied torque at each joint, the combination of joint angles (θ) measured by the IMU sensors and the actuation pressure measured by the pressure sensors are used through a theoretical model, such as the model described in aforementioned U.S. Patent Application No. US 2018/0303698. These loops control the angular motion of the finger joints and the force exerted from the robotic digit and human finger onto an object. The final control action (an air pressure, Pₐ) is then determined by a combination of actuation pressure or vacuum associated with the position (P_{θ}), force (P_{f}), and current internal pressure (P) of actuators measured by in-line pressure sensors. In order to ensure safe operation of the actuators interacting with the hand, a set of safety check conditions can be implemented in the control algorithms that avoid the occurrence of unwanted physiological reactions.

Since tuning the control gains for achieving a desired performance according to each user's biomechanical characteristics is not an intuitive task, an adaptive control algorithm can be integrated into the force-position controller, as shown in FIG. 6. The proportional-integral-derivative (PID) control gains can be adaptively adjusted using a set of adaptive laws based on the error between the desired and actual position and force in order to achieve the desired dynamic responses from the robotic digits in interaction with fingers and external objects based on the user's condition and therapeutic tasks.

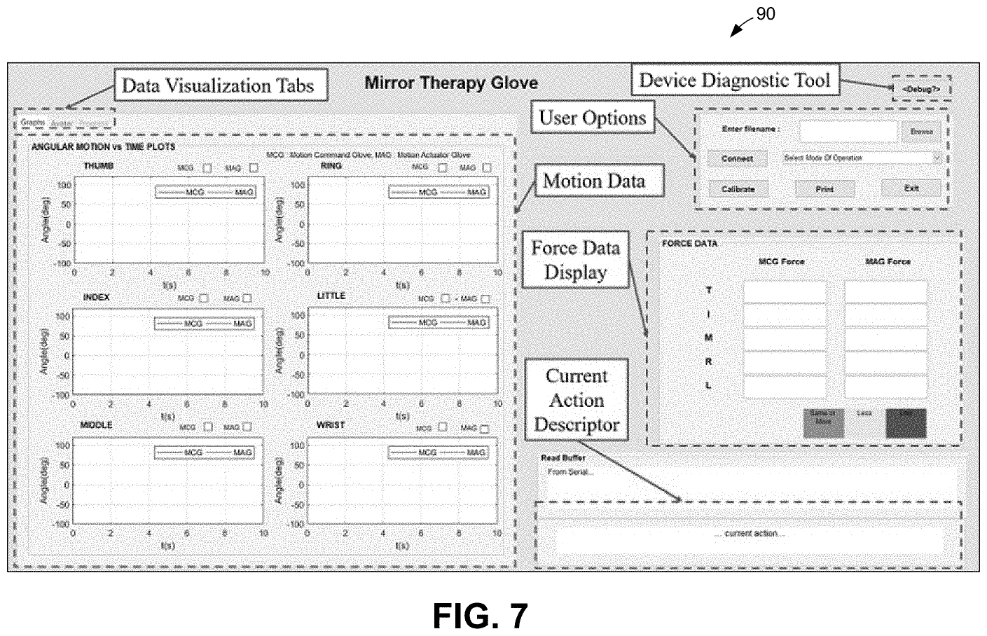

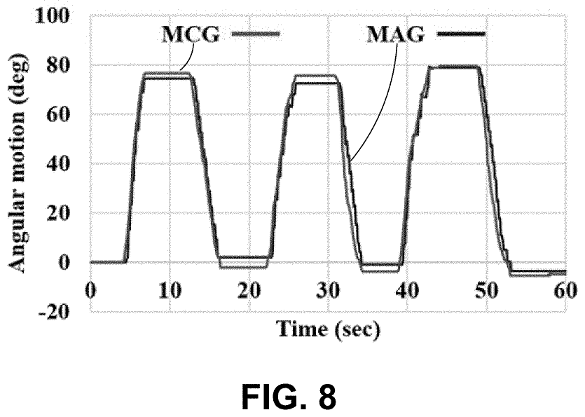

As mentioned above, various information can be conveyed to the user and/or a medical professional using a graphical user interface (GUI). FIG. 7 illustrates an example of such a GUI 90. This GUI 90 has three main functions: (1) motion data visualization, (2) force level indication, and (3) user option selection. A Data Visualization tab in the GUI 90 can present data in three forms: graphical plots, hand avatar, and progress. As shown in FIG. 7, the Data Visualization tab can display a graphical representation of the angular motion of the user's joints. The plotting area can include graphs related to the angular positions of the thumb, index, middle, ring, little, and wrist. Each graph can contain data from both the MCG 12 and the MAG 14 for purposes of comparison. FIG. 8 illustrates an example of such a graph. In this graph, an angular motion of the MCG 12 (e.g., rotation of a joint) is compared to angular motion of a corresponding part of the MAG 14. As indicated in the graph, there is close agreement between movement of the MCG 12 and the MAG 14 as a function of time. In some embodiments, hand avatars can be displayed in real time to the user during a rehabilitation session to graphically represent via animation the motion of the user's hands. With reference back to FIG. 7, a Progress Report tab of the GUI 90 displays the daily, weekly, or monthly therapy progress in terms of joint range of motion and grip strength.

A Force Data Display of the GUI 90 presents the data received by the GUI and can use a color scheme to indicate the closeness of the force applied by the hand with MAG 12 to the force captured from the MCG 14. For example, red can be used to depict when the force is much lower than the required force, yellow can be used to depict when the force has increased but is still lower than the required force, and green can be used to depict when the force required to perform the desired motion equals that required for the manipulation task.

The User Options section of the GUI 90 includes a record button for data recording, a connect button that connects the GUI to the MAG 12 and the MCG 14, and a select button for selecting one of various operation modes, including gesture, single-hand manipulation, and two-hand manipulation. In addition, the User Options section contains a calibrate button that can be used to calibrate the system before the activity begins. In addition, there is a print button for printing desired data and an exit button for terminating the therapy session. A Current Activity Descriptor section of the GUI 90 provides information as to what activity is taking place at the time, whether it is simple flexion/extension, grasping, or manipulation.

1. A robotic mirror therapy system for providing therapy to a user having a impaired hand, the system comprising:
a motion command glove configured to be worn on a healthy hand of the user or another person, the motion command glove comprising position sensors configured to determine the position and orientation of the healthy hand and its fingers;
a motion actuator glove configured to be worn on the user's impaired hand, the motion actuator glove comprising actuators configured to actuate the impaired hand and its fingers; and
a control unit configured to receive position data from the motion command glove and to control actuation of the actuators of the motion actuator glove such that the impaired hand mirrors motion of the healthy hand.

2. The system of claim 1, wherein the position sensors comprise inertial measurement units.

3. The system of claim 2, wherein the system comprises an inertial measurement unit for each segment of each finger of the healthy hand.

4. The system of claim 1, wherein the position sensors comprise flex sensors.

5. The system of claim 4, wherein the system comprises a bend sensor for each finger of the healthy hand.

6. The system of claim 1, wherein the actuators are fluidic actuators. The system of claim 6, wherein the actuators are pneumatic actuators.

8. The system of claim 6, wherein the control unit comprises a pneumatic system configured to alter a pressure of a drive fluid used to drive the fluidic actuators, the pneumatic system including a pump, valves, and pressure sensors.

9. The system of claim 1, wherein the motion command glove further comprises force sensors configured to measure forces applied to the fingers and palm of the healthy hand and wherein data collected by the force sensors is also provided to the control unit.

10. The system of claim 1, wherein the motion actuator glove further comprises position sensors configured to determine the position and orientation of the impaired hand and its fingers and wherein data collected by the position sensors of the motion actuator glove is also provided to the control unit.

11. The system of claim 1, wherein the motion actuator glove further comprises force sensors configured to measure forces applied to the fingers and palm of the impaired hand and wherein data collected by the force sensors is also provided to the control unit.

12. The system of claim 1, further comprising a robotic mirror therapy program that controls operation of the control unit.

13. The system of claim 12, wherein the robotic mirror therapy program receives data collected by the motion command glove and the motion actuator glove and wherein the program analyzes the data to determine how to control the actuators of the motion actuator glove.

14. The system of claim 12, wherein the robotic mirror therapy program comprises a non-adaptive force-position control algorithm configured to control actuation of the actuators of the motion actuator glove based on position and force data received from the position sensors of the motion control glove.

15. The system of claim 12, wherein the robotic mirror therapy program comprises an adaptive force-position control algorithm configured to control actuation of the actuators of the motion actuator glove based on position and force data received from the position sensors of the motion control glove.

16. The system of claim 1, further comprising a graphical user interface configured to display data collected by the system.

17. A motion command glove configured to be worn on a healthy hand, the glove comprising:
finger sleeves configured to be placed over each finger of the healthy hand, each finger sleeve having a dorsal side and a palmer side;
at least one position sensor provided on the dorsal side of each finger sleeve; and
at least one force sensor provided on the palmar side of each finger sleeve.

18. A motion actuator glove configured to be worn on an impaired hand, the glove comprising:
finger sleeves configured to be placed over each finger of the impaired hand, each finger sleeve having a dorsal side and a palmer side;
at least one actuator provided on the dorsal side of each finger sleeve;
at least one position sensor provided on the dorsal side of each finger sleeve; and
at least one force sensor provided on the palmar side of each finger sleeve.

19. A method for providing robotic mirror therapy to an impaired hand of a user, the method comprising:
tracking motions of a healthy hand with a motion command glove worn on the healthy hand, the motion command glove including at least one position sensor associated with each finger of the healthy hand; and
actuating a motion actuator glove worn on a user's impaired hand so as to mirror the motions of the healthy hand, the motion actuator glove including a soft actuator associated with each finger of the impaired hand;
wherein a robotic mirror therapy program uses an adaptive or non-adaptive force-position control algorithm to determine how to actuate the actuators of the motion actuator glove responsive to position data received from the motion command glove.

20. The method of claim 19, wherein the position sensors comprise inertial measurement units or bend sensors and the actuators comprise pneumatic actuators.
